(19) United States
(12) Patent Application Publication
(10) Pub. No.: US 2024/0099588 A1
(43) Pub. Date: Mar. 28, 2024

(54) APPARATUS FOR INPUTTING COMBINED IMAGE OF PHOTOACOUSTIC IMAGE AND ULTRASONIC IMAGE AND METHOD THEREOF

(71) Applicant: PUKYONG NATIONAL UNIVERSITY INDUSTRY-UNIVERSITY COOPERATION FOUNDATION, Busan (KR)

(72) Inventor: Jung Hwan OH, Busan (KR)

(73) Assignee: PUKYONG NATIONAL UNIVERSITY INDUSTRY-UNIVERSITY COOPERATION FOUNDATION, Busan (KR)

(21) Appl. No.: 18/220,851

(22) Filed: Jul. 12, 2023

(30) Foreign Application Priority Data
Sep. 27, 2022 (KR) 10-2022-0122802

(51) Int. Cl. A61B 5/00

(52) U.S. Cl. CPC A61B 5/0095

(57) ABSTRACT

Provided is an apparatus for inputting a combined photoacoustic/ultrasonic image, according to an embodiment of the present disclosure, which generates three-dimensional image for a to-be-examined object by performing two-dimensional scanning on the to-be-examined object the apparatus comprising: a photoacoustic probe that outputs a laser pulse output or an ultrasonic output to a to-be-examined object, receives a first ultrasonic input from the to-be-examined object by the laser pulse output, and receives a second ultrasonic input from the to-be-examined object by the ultrasonic output; an ultrasonic transceiving unit that generates an ultrasonic output signal for generating the ultrasonic output, receives the first ultrasonic input and the second ultrasonic input, and generates a photoacoustic image signal and an ultrasonic image signal, respectively; and a main controller that generates photoacoustic image and ultrasonic image for the to-be-examined object, and combines the photoacoustic image and ultrasonic image to generate a combined photoacoustic/ultrasonic image.

### CROSS-REFERENCE TO RELATED APPLICATION

This application claims priority to and the benefit of Korean Patent Application No. 10-2022-0122802 filed on Sep. 27, 2022, in the Korean Intellectual Property Office, the entire disclosure of which is incorporated herein by reference.

### BACKGROUND

### 1. Field

The present disclosure relates to an apparatus for inputting a combined image of a photoacoustic image and an ultrasonic image and a method thereof. More specifically, the present disclosure relates to an apparatus for inputting a combined image of a photoacoustic image and an ultrasonic image and a method thereof, in which a combined photoacoustic/ultrasonic image in which the photoacoustic and ultrasonic images for the inside of a to-be-examined object are combined can be generated, while moving a photoacoustic probe at high speed.

### 2. Description of the Related Art

When light having very high energy is irradiated to a to-be-examined object, the to-be-examined object that absorbs the light energy thermally expands elastically. Due to this elastic expansion, a pressure wave is generated, and the generated pressure wave takes the form of an ultrasonic wave. This phenomenon is called a “photo-acoustic effect”, and an ultrasonic signal generated by this expansion is called a photoacoustic signal.

Recently, a technique for acquiring state information of an object, particularly the inside of the to-be-examined object, by using a photoacoustic effect, and generating the same as image information, has been spotlighted. Particularly in the medical field, a lot of research on this is being done. In the medical field, during a disease treatment process, it is often necessary to visually check state information about the inside of a living body. Currently, tools that are widely used as a method for generating image information about the inside of a living body include X-ray, CT, MRI, etc., as is well known. However, it has been reported that these methods are accompanied by various problems, including expensive equipment, an extremely low resolution of a generated image, a narrow field of view (FOV), an extended time required to implement the image, or possibility of harming the human body due to continuous use.

A method for generating a photoacoustic image of an internal state of a living body by using a photoacoustic effect is attracting attention as an alternative to these methods. In particular, the photoacoustic image can show blood vessel-related information concerning the inside of the human body, and thus can be an important technology in the medical field.

Meanwhile, an ultrasonic system may generate an ultrasonic image by outputting an ultrasonic signal to a to-be-examined object and receiving an ultrasonic signal output from the to-be-examined object. In particular, the ultrasonic system has non-invasive and non-destructive properties for an object, and is widely used in various fields. Recently, an ultrasonic system is used to generate a 2D or 3D image of the internal shape of an object. In particular, ultrasonic images can show information related to the structure of a human body.

A photoacoustic image can mainly show only blood vessel-related information, and an ultrasonic image can mainly show only structure-related information. Accordingly, there is a need for a technology capable of simultaneously displaying structure-related information and blood vessel-related information for the inside of a human body.

### SUMMARY

The present disclosure provides an apparatus for inputting a combined image of a photoacoustic image and an ultrasonic image, and a method thereof, in which a combined photoacoustic/ultrasonic image in which the photoacoustic and ultrasonic images for the outside and/or the inside of a to-be-examined object are combined can be generated, while moving a single photoacoustic probe at high speed.

Provided is an apparatus for inputting a combined photoacoustic/ultrasonic image (hereinafter, to be also briefly referred to as a combined photoacoustic/ultrasonic image input apparatus), according to an embodiment of the present disclosure, which generates three-dimensional (3D) image information for a to-be-examined object by performing two-dimensional (2D) scanning on the to-be-examined object by a first-direction linear motion of a photoacoustic probe and a second-direction linear motion that is substantially perpendicular to the first-direction linear motion, the apparatus comprising: a photoacoustic probe that outputs a laser pulse output or an ultrasonic output to a to-be-examined object, receives a first ultrasonic input from the to-be-examined object by the laser pulse output, and receives a second ultrasonic input from the to-be-examined object by the ultrasonic output; an ultrasonic transceiving unit that generates an ultrasonic output signal for generating the ultrasonic output, receives the first ultrasonic input and the second ultrasonic input, and generates a photoacoustic image signal and an ultrasonic image signal, respectively; an analog/digital (ND) converter that receives the photoacoustic image signal and the ultrasonic image signal to convert the same into digital image signals; and a main controller that receives the digital image signals, generates photoacoustic image information and ultrasonic image information for the to-be-examined object, and combines the photoacoustic image information and ultrasonic image information to generate a combined photoacoustic/ultrasonic image.

The photoacoustic probes may include a laser output unit that outputs the laser pulse output to the to-be-examined object, and an ultrasonic probe that outputs the ultrasonic output to the to-be-examined object and receives the first ultrasonic input and the second ultrasonic input, respectively.

The apparatus may include: a pulse signal generator that generates and outputs a reference pulse signal at a set interval; a first linear encoder that generates first-direction linear motion information of the photoacoustic probe; a laser generator that outputs a laser pulse at a set interval to the to-be-examined object according to a reference pulse signal and the first-direction linear motion information; and a trigger controller that generates an output trigger signal at a set interval according to the reference pulse signal and the first-direction linear motion information.

The ultrasonic transceiving unit may generate the photoacoustic image signal and the ultrasonic image signal corresponding to the first-direction linear motion information according to the output trigger signal, respectively.

The ultrasonic probe may output an ultrasonic output corresponding to an output trigger signal generated by the trigger controller and receive the ultrasonic input corresponding to the first-direction linear motion information according to the output trigger signal.

In the main controller, photoacoustic image information for the to-be-examined object may be generated by sequentially combining the photoacoustic digital image signal with the photoacoustic image information corresponding to each trigger pulse of the output trigger signal in a positive direction or a negative direction of the first direction in units of photoacoustic digital image scan lines, and ultrasonic image information for the to-be-examined object may be generated by sequentially combining the ultrasonic digital image signal with the ultrasonic image information corresponding to each trigger pulse of the output trigger signal in the positive or negative direction of the first direction in units of scan lines.

The photoacoustic image information may be generated by first two-dimensional (2D) scanning from a first scan line to an n-th scan line, and the ultrasonic image information may be generated by second 2D scanning from the first scan line to the n-th scan line.

The photoacoustic image information may be generated by first 2D scanning from an n-th scan line to a first scan line, and the ultrasonic image information may be generated by second 2D scanning from the n-th scan line to the first scan line.

While moving in a first direction from a first end to a second end of the n-th scan line, the photoacoustic image information may be generated by a positive (+) first-direction motion of the photoacoustic probe, and, without moving the photoacoustic probe in a second direction, while moving in the first direction from the first end to the second end of the n-th scan line, the ultrasonic image information may be generated by the positive (+) first-direction motion of the photoacoustic probe.

While moving in the first direction from the first end to the second end of the n-th scan line, the photoacoustic image information may be generated by the positive (+) first-direction motion of the photoacoustic probe, and, without moving the photoacoustic probe in the second direction, while moving in a direction opposite to the first direction from the second end to the first end of the n-th scan line, the ultrasonic image information may be generated by a reverse motion (negative (−) first-direction motion) to the first-direction motion of the photoacoustic probe.

The apparatus may further include an output selector for selecting the laser pulse output or the ultrasonic output to be output.

The apparatus may further include an output selector for selecting a reference pulse signal to be output to the trigger controller or the laser generator.

The main controller may generate and output an output selection signal, and the pulse signal generator may output the reference pulse signal to the trigger controller or the laser generator according to the output selection signal.

While moving the photoacoustic probe in the first direction, which is a direction from the first end to the second end of the n-th scan line, the laser pulse output and the ultrasonic output may be alternately performed in the photoacoustic probe.

Three-dimensional (3D) image information for the to-be-examined object may be generated by one-time 2D scanning of the photoacoustic probe, and the first ultrasonic input and the second ultrasonic input may be alternately performed within each scanning line.

The apparatus may further include: a second linear encoder for generating the second-direction linear motion information of the photoacoustic probe; and a memory for storing plane coordinate values of the photoacoustic probe, determined by the first-direction linear motion information and the second-direction linear motion information, photoacoustic image information at the plane coordinate values, and ultrasonic image information for the plane coordinate values of the photoacoustic probe and the to-be-examined object at the plane coordinate values.

With respect to the to-be-examined object, the photoacoustic image information may be generated by 2D scanning while allowing the photoacoustic probe to alternately perform the first-direction motion and the second-direction motion from start coordinates to end coordinates, and, after completing the 2D scanning, the ultrasonic image information may be generated by the 2D scanning while allowing the photoacoustic probe to alternately perform the first-direction motion and the second-direction motion from the start coordinates to the end coordinates.

With respect to the n-th scan line, the photoacoustic image information may be received while moving the photoacoustic probe in the first direction, and the ultrasonic image information may be received while moving the photoacoustic probe in a direction opposite to the first direction, and, with respect to an (n+1)-th scan line, the photoacoustic image information may be received while moving the photoacoustic probe in the first direction, and the ultrasonic image information may be received while moving the photoacoustic probe in a direction opposite to the first direction.

With respect to the n-th scan line, the photoacoustic image information and the ultrasonic image information may be alternately received while moving the photoacoustic probe in the first direction, and, with respect to an (n+1)-th scan line, the photoacoustic image information and the ultrasonic image information may be alternately received while moving the photoacoustic probe in a direction opposite to the first direction the photoacoustic image information.

Alternatively, in a method for inputting a combined photoacoustic/ultrasonic image, according to an embodiment of the present disclosure, a combined photoacoustic/ultrasonic image may be acquired by using the combined photoacoustic/ultrasonic image input apparatus 1 according to the above-described method.

### BRIEF DESCRIPTION OF DRAWINGS

FIG. 1 is a block diagram schematically showing an apparatus for inputting a combined image of a photoacoustic image and an ultrasonic image, according to an embodiment of the present disclosure.

FIG. 2 is a conceptual diagram schematically showing an optical-resolution apparatus for inputting a combined image of a photoacoustic image and an ultrasonic image, according to another embodiment of the present disclosure.

FIG. 3 is a timing diagram schematically showing a method for generating a combined image by a photoacoustic image signal in the combined photoacoustic/ultrasonic image input apparatus shown in FIG. 1.

FIG. 4 is a timing diagram schematically showing a method for generating a combined image by an ultrasonic image signal in the combined photoacoustic/ultrasonic image input apparatus shown in FIG. 1.

### DETAILED DESCRIPTION

Hereinafter, specific details for implementing the present disclosure will be described in detail with reference to the accompanying drawings based on preferred embodiments of the present disclosure. Here, as disclosed in the drawing of one embodiment, the same reference numerals are assigned to the same components as those disclosed in the drawings of another embodiment, and descriptions in other embodiments may be equally applied, and detailed description thereof is simplified or omitted here. In addition, known functions or configurations related to the present disclosure refer to known technologies, and detailed description thereof is simplified or omitted here.

In addition, although the terms used in this specification are selected from generally known and used terms, the terms used herein may be varied depending on operator's intention or customs in the art, advent of new technology, or the like. In addition, in a specific case, some of the terms mentioned in the description of the present disclosure have been arbitrarily selected by the inventor(s), and, in this case, the detailed meanings of the terms will be described in detail in relevant parts of the description of the present disclosure. Therefore, the terms used in this specification should be defined based on the meanings of the terms and the overall content of the present disclosure, not simply on the basis of actual terms used.

Throughout the specification, it will be understood that the term “comprising or including” specifies the addition and/or presence of one or more other components, but does not preclude the possibility of excluding the stated components features, unless the context clearly indicates otherwise. In addition, the term “unit” used herein refers to software or a hardware element such as a field-programmable gate array (FPGA), an application specific integrated circuit (ASIC), etc. However, the meaning of “unit” is not limited to software or hardware. The “unit” may advantageously be configured to reside on the addressable storage medium and configured to drive one or more processors. Thus, a unit may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and “unit(s)” may be combined into fewer components and “unit(s)” or further separated into additional components and “unit(s)”.

A method for generating a photoacoustic image of an object, for example, the inside of a living body by using a photoacoustic effect will now be described. First, an optical beam (e.g., a laser beam) is irradiated to a specific part of a living body, from which a 3D image is intended to acquire, a photoacoustic signal generated according to thermal elastic expansion generated in the specific part by the irradiated beam (an ultrasonic signal) is acquired through an ultrasonic probe (ultrasonic transducer), and the acquired photoacoustic signal is subjected to predetermined signal processing to generate 3D photoacoustic image information for the inside of the living body.

In addition, a method for generating an ultrasonic image for the inside of a to-be-examined object will now be described. First, an ultrasonic beam is irradiated to a specific part of a living body, from which a 3D image is intended to acquire, an ultrasonic signal generated in the specific part by the irradiated ultrasonic beam is acquired through an ultrasonic probe (ultrasonic transducer), and the acquired ultrasonic signal is subjected to predetermined signal processing to generate 3D ultrasonic image information for the inside of the living body.

A high-speed scanning photoacoustic image input apparatus according to an embodiment of the present disclosure may include a photoacoustic microscope (PAM). In addition, a photoacoustic probe for a photoacoustic microscope (PAM) may scan a target area including a to-be-examined object while moving at high speed by using a slider crank mechanism. The high-speed scanning photoacoustic image acquiring apparatus may convert a unidirectional rotational motion of a driving motor into a linear reciprocating motion of the photoacoustic probe connected to the driving motor. In addition, a three-dimensional (3D) image of the to-be-examined object may be generated by two-dimensional (2D) scanning of the to-be-examined object by the linear motion of the photoacoustic probe and the vertical motion perpendicular to the linear motion.

In the photoacoustic microscope (PAM) of the present disclosure, an optical-resolution PAM (OR-PAM) having micron-scale spatial resolution by focusing an optical beam (e.g., a laser beam) may be used. The optical-resolution PAM (OR-PAM) may use a tight optical focus. Meanwhile, the acoustic-resolution PAM (AR-PAM) may use an acoustic focus.

Since the optical-resolution PAM (OR-PAM) relies on an optical beam that is much tighter than an acoustic beam, there is an advantage in that high-resolution images can be acquired, compared to an acoustic-resolution PAM (AR-PAM). In addition, the optical-resolution PAM (OR-PAM) has rich optical absorption contrast, and thus can be a powerful imaging tool in most fields related to medicine, including many fields such as biology, dermatology, neurology, oncology, ophthalmology, pathology, and so on.

To achieve maximization of a signal-to-noise ratio (SNR) and optimization of spatial resolution, the optical-resolution PAM (OR-PAM) may apply confocal and coaxial configurations of optical excitation beams and acoustic detection beams. Volumetric imaging is typically achieved by point-by-point raster scanning of optical and acoustic beams, for which a stepping motor scanning stage may be applied.

Because of the scanning step size required by micron-level lateral resolution, the scanning speed (and consequent imaging speed) and scanning range of the optical-resolution PAM (OR-PAM) may be low (B-scan rate of approximately 1 Hz in a scanning range of 1 mm). Due to such a low imaging speed, it has not been easy to acquire tissue's dynamic information such as transient drug response or skin vasculature by an optical-resolution PAM (OR-PAM).

Meanwhile, in the optical-resolution PAM (OR-PAM), there may be various methods for improving the field of view (FOV) corresponding to a scanning range, increasing the scanning speed or shortening the scanning time, and maintaining a high signal-to-noise ratio (SNR). In order to implement an optical-resolution PAM (OR-PAM), trade-offs of these three characteristics are required, and these trade-offs may act as a factor that makes it difficult to implement the optical-resolution PAM (OR-PAM) that satisfies all three characteristics. This is because a required scanning time depends on a pulse repetition rate of laser and a scanning mechanism, and is limited by the sound speed of a photoacoustic (PA) wave in a tissue.

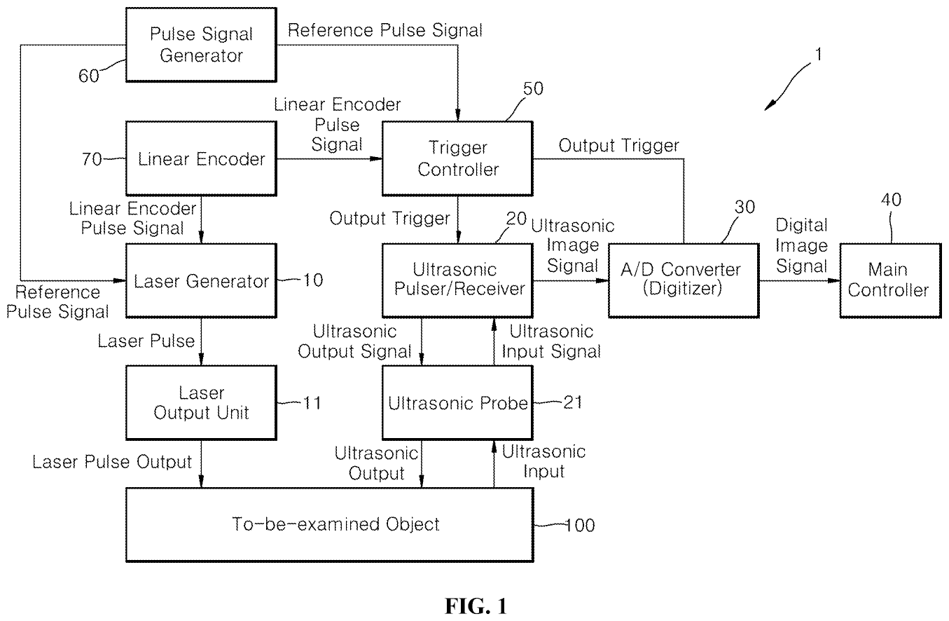

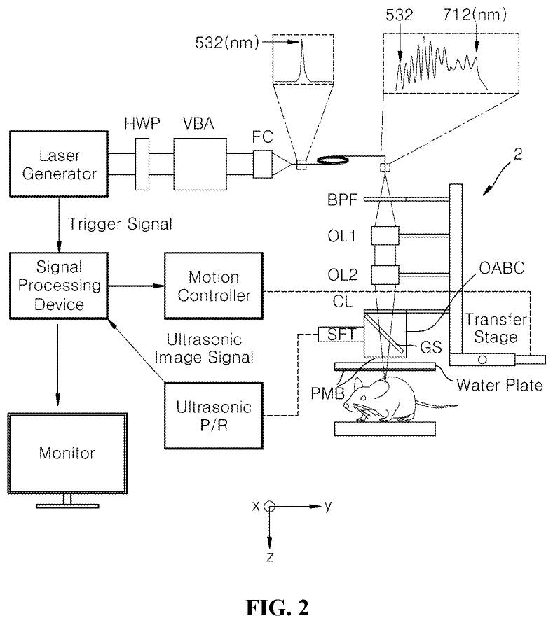

FIG. 1 is a block diagram schematically showing an apparatus for inputting a combined photoacoustic/ultrasonic image, according to an embodiment of the present disclosure. FIG. 2 is a conceptual diagram schematically showing an optical-resolution apparatus for inputting a combined photoacoustic/ultrasonic image, according to another embodiment of the present disclosure.

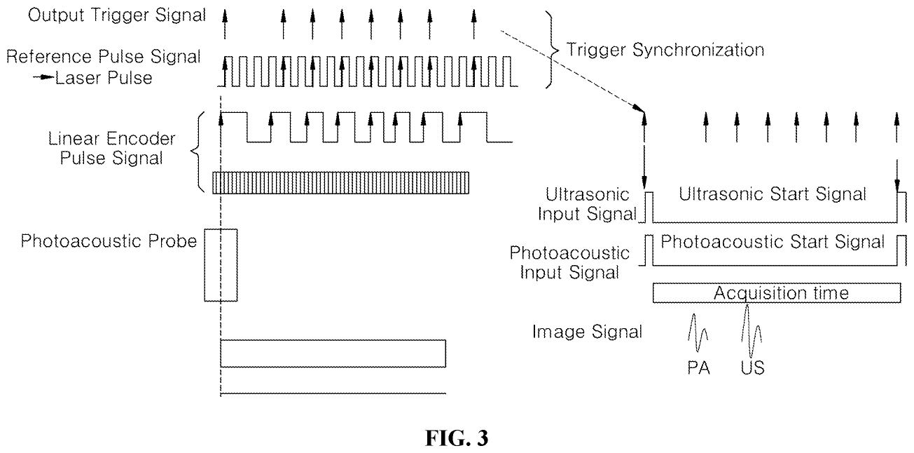

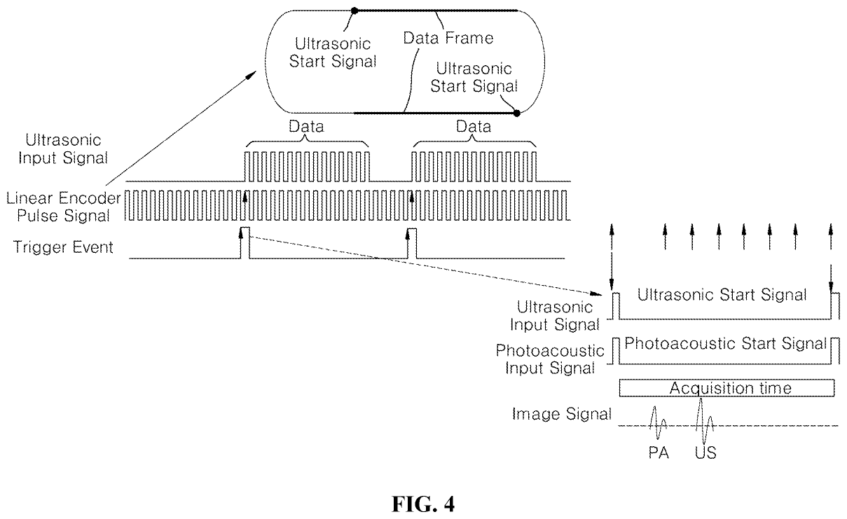

FIG. 3 is a timing diagram schematically showing a method for generating a combined image by a photoacoustic image signal in the combined photoacoustic/ultrasonic image input apparatus shown in FIG. 1. FIG. 4 is a timing diagram schematically showing a method for generating a combined image by an ultrasonic image signal in the combined photoacoustic/ultrasonic image input apparatus shown in FIG. 1.

Referring to the drawings, the combined photoacoustic/ultrasonic image input apparatus 1 may generate a 3D combined photoacoustic/ultrasonic image for a to-be-examined object, by performing 2D scanning on the to-be-examined object by a first-direction linear motion of one photoacoustic probe 11, 21 and a second-direction linear motion that is substantially perpendicular to the first-direction linear motion. According to the present disclosure, a photoacoustic and ultrasonic combined image combining a photoacoustic image and an ultrasonic image may be generated by using only a photoacoustic probe without a separate ultrasonic module.

The combined photoacoustic/ultrasonic image input apparatus 1 may include: a photoacoustic probe 11, 21; an ultrasonic transceiving unit 20; an analog/digital (A/D) converter 30; and a main controller 40.

The photoacoustic probes 11 and 21 may output a laser pulse output or an ultrasonic output to the to-be-examined object, may receive a first ultrasonic input output from the to-be-examined object by the laser pulse output, and may receive a second ultrasonic input output from the to-be-examined object by the ultrasonic output. Here, the first ultrasonic input may be a signal received by inputting an ultrasonic signal output from the to-be-examined object as the laser pulse output is input to the to-be-examined object. The second ultrasonic input may be a signal received by inputting an ultrasonic pulse output from the to-be-examined object as the ultrasonic pulse output is input to the to-be-examined object.

The ultrasonic transceiving unit 20 may generate an ultrasonic output signal for generating an ultrasonic output, receive a first ultrasonic input and a second ultrasonic input, respectively, and generate a photoacoustic image signal and an ultrasonic image signal, respectively. In this case, the photoacoustic image signal may be generated from the first ultrasonic input, and the ultrasonic image signal may be generated from the second ultrasonic input. The ultrasonic transceiving unit 20 may include a pulser/receiver that generates and outputs an ultrasonic pulse through the ultrasonic probe 21 and receives an ultrasonic signal reflected from the to-be-examined object through the ultrasonic probe 21, and may further include an amplifier for amplifying the ultrasonic signal.

The A/D converter 30 may receive a photoacoustic image signal and an ultrasonic image signal from the ultrasonic transceiving unit 20 and convert the same into digital image signals, respectively. Here, the digital image signals may include a digital photoacoustic image in which an analog photoacoustic image signal is digitally converted and a digital ultrasonic image signal in which an analog ultrasonic image signal is digitally converted.

The main controller 40 may receive the digital photoacoustic image signal and the digital ultrasonic image signal from the A/D converter 30, respectively, to generate photoacoustic image information and ultrasonic image information of the to-be-examined object, and may combine the photoacoustic image information and the ultrasonic image information to generate a combined photoacoustic/ultrasonic image. Here, each of the digitally converted photoacoustic image information and ultrasonic image information may include location information of the to-be-examined object and digital image information corresponding to the location information.

Therefore, the main controller 40 may generate photoacoustic image information and ultrasonic image information corresponding to each location information in the to-be-examined object and combine the same to generate a combined photoacoustic/ultrasonic image corresponding to each location information. The main controller 40 may simultaneously display the photoacoustic image and the ultrasonic image of the to-be-examined object on one display.

In this case, a user can check the photoacoustic image and the ultrasonic image of the to-be-examined object at once through a monitor. Accordingly, the combined photoacoustic/ultrasonic image input apparatus 1 can simultaneously display structure-related information and blood vessel-related information for the inside of a human body through the combined photoacoustic/ultrasonic image. That is, the user can check information related to blood vessels through the photoacoustic image on one screen and at the same time check information related to a structure through the ultrasonic image.

The photoacoustic probes 11 and 21 may include a laser output unit 11 and an ultrasonic probe 21. The laser output unit 11 may output the laser pulse output generated from the laser generator 10 to the to-be-examined object, and the ultrasonic probe 21 may output an ultrasonic pulse to the to-be-examined object or may receive an ultrasonic signal from the to-be-examined object.

In addition, the ultrasonic transceiving unit 20 may output the generated ultrasonic output signal to output the ultrasonic output to the to-be-examined object through the ultrasonic probe 21, or may receive the ultrasonic signal input from the to-be-examined object through the ultrasonic probe 21. In this case, the ultrasonic output may be an ultrasonic pulse output.

The ultrasonic transceiving unit 20 may include a pulser that generates and outputs an ultrasonic pulse signal through the ultrasonic probe 21 and a receiver that receives an ultrasonic signal generated from the to-be-examined object through the ultrasonic probe 21. That is, by including an ultrasonic pulser capable of outputting ultrasonic pulses, the ultrasonic transceiving unit 20 may output a separate ultrasonic pulse to the to-be-examined object through the ultrasonic probe 21, unlike in a conventional photoacoustic input device.

In addition, the ultrasonic input received from the to-be-examined object may be a first ultrasonic input generated from the to-be-examined object by a laser pulse output or a second ultrasonic input generated from the to-be-examined object by an ultrasonic pulse output.

That is, in the combined photoacoustic/ultrasonic image input apparatus 1, according to an embodiment of the present disclosure, unlike in a general photoacoustic probe in which an ultrasonic signal output from the to-be-examined object is received and transmitted to an ultrasonic receiver, the ultrasonic probe 21 included in the photoacoustic probes 11 and 21 may output the ultrasonic output according to the ultrasonic output signal generated from the ultrasonic transceiving unit 20 to the to-be-examined object.

Therefore, the ultrasonic probe 21 according to an embodiment of the present disclosure may generate a photoacoustic image by receiving the ultrasonic signal generated by the to-be-examined object according to the ultrasonic signal laser pulse output through the ultrasonic transceiving unit 20, and may output the ultrasonic output according to the ultrasonic output signal generated by the ultrasonic transceiving unit 20 to the to-be-examined object, and thus may generate an ultrasonic image by receiving the ultrasonic signal generated in the to-be-examined object through the ultrasonic transceiving unit 20.

Here, in the combined photoacoustic/ultrasonic image input apparatus 1, according to an embodiment of the present disclosure, the ultrasonic probe 21 may separately receive the first ultrasonic input and the second ultrasonic input by distinguishing the input timing of the ultrasonic signal (first ultrasonic input) generated in the to-be-examined object according to the laser pulse output from the input timing of the ultrasonic signal (second ultrasonic input) generated in the to-be-examined object according to the ultrasonic pulse output.

To this end, by distinguishing the output timing of the laser pulse output from the output timing of the ultrasonic pulse output, it is possible to prevent signal interference between the first ultrasonic input and the second ultrasonic input.

Therefore, in the combined photoacoustic/ultrasonic image input apparatus 1, unlike in a general photoacoustic image input apparatus in which ultrasonic signals are merely received through an ultrasonic probe, the ultrasonic probe 21 and the ultrasonic transceiving unit 20 are allowed to output ultrasonic images, and thus, by distinguishing timings just by means of a photoacoustic probe, both photoacoustic images and ultrasonic images can be received, even without including a separate ultrasonic probe for receiving ultrasonic images.

To this end, as shown in FIG. 1, the combined photoacoustic/ultrasonic image input apparatus 1 may include a laser generator 10, a ultrasonic transceiving unit 20, photoacoustic probes 11 and 21, an analog/digital (A/D) converter 30, a main controller 40, a trigger controller 50, a pulse signal generator 60, and a linear encoder 70.

The pulse signal generator 60 may generate and output a reference pulse signal at a set interval (e.g., a certain time interval). The linear encoder 70 may include a first linear encoder that generates linear motion information in a first direction and a second linear encoder that generates linear motion information in a second direction that is substantially perpendicular to the first direction. The linear encoder 70 may generate a linear encoder pulse signal corresponding to the first-direction linear motion information of the photoacoustic probes 11 and 21.

The laser generator 10 may output a laser pulse at a set interval (e.g., certain positions and/or time intervals) in the to-be-examined object according to the reference pulse signal and the linear encoder pulse signal corresponding to the first-direction linear motion information. As shown in FIG. 3, the laser pulse may be generated such that the linear encoder pulse signal is synchronized with the reference pulse signal after being input.

In this case, since the laser generator 10 outputs the laser pulse according to the linear encoder pulse signal and the reference pulse signal generated by the pulse signal generator 60, the photoacoustic image information corresponding to accurate position information can be generated without a separate scanning trigger.

Meanwhile, the trigger controller 50 may generate an output trigger signal at (e.g., certain positions and/or time intervals) according to the reference pulse signal and the linear encoder pulse signal corresponding to the first-direction linear motion information. As shown in FIGS. 3 and 4, in a corresponding scanning line (n-th scanning line), the first output trigger signal may be used as an ultrasonic start signal, and, after the ultrasonic start signal is input, a preset number of ultrasonic inputs may be made. In this case, as shown in FIG. 3, since the output trigger signal is generated in synchronization with the linear encoder pulse signal and the reference pulse signal, ultrasonic image information according to the ultrasonic input may include ultrasonic image information derived according to accurate position information.

The ultrasonic transceiving unit 20 may generate an ultrasonic output signal corresponding to the output trigger signal, and the ultrasonic probe 21 irradiates the to-be-examined object 100 with an ultrasonic pulse output according to the ultrasonic output signal. Accordingly, an ultrasonic input may be output from the to-be-examined object 100, the ultrasonic input may be received from the ultrasonic transceiving unit 20 through the ultrasonic probe 21, and a digital ultrasonic image signal may be output to the A/D converter 30. The main controller 40 may receive the digital ultrasonic image signal, combine the same with each location information to generate an ultrasonic image, and combine the same with the photoacoustic image generated by being combined with each location information to generate a combined image.

Here, the ultrasonic transceiving unit 20 may generate a photoacoustic image signal and an ultrasonic image signal corresponding to the first-direction linear motion information according to the output trigger signal, respectively, and a combined image of the photoacoustic image signal and the ultrasonic image signal may be generated at the correct position.

The ultrasonic probe 21 may output an ultrasonic output corresponding to the output trigger signal generated by the trigger controller 50, and may receive the ultrasonic input corresponding to the first-direction linear motion information according to the output trigger signal.

The main controller 40 may sequentially combine photoacoustic image information corresponding to each trigger pulse of the output trigger signal in a positive or negative direction of the first direction in units of scan lines to generate the photoacoustic image information for the to-be-examined object, may sequentially combine the ultrasonic image information corresponding to each trigger pulse of the output trigger signal in the positive or negative direction of the first direction in units of the same scan line to generate the ultrasonic image information for the to-be-examined object, and may generate a combined image by combining the photoacoustic image information and the ultrasonic image information according to the position information (a linear encoder pulse signal) included in the output trigger signal within the same scan line.

Meanwhile, the linear encoder pulse signal may be a pulse signal output from the linear encoder 70 or a signal corresponding to an integer multiple of the pulse signal.

As an embodiment, photoacoustic image information may be generated by performing first 2D scanning from a first scan line to an n-th scan line. Then, after the photoacoustic probes 11 and 21 move to a first end or a second end of the first scan line, the ultrasonic image information may be generated by performing second 2D scanning from the first scan line to the n-th scan line.

Here, for the to-be-examined object 100, the photoacoustic probes 11 may generate photoacoustic image information while performing 2D scanning while alternately moving in the first direction and moving in the second direction from start coordinates to end coordinates, and after completing the first 2D scanning, the photoacoustic probes 11 and 21 may generate ultrasonic image information by performing 2D scanning while alternately moving in the first direction and moving in the second direction from the start coordinates to the end coordinates.

As another embodiment, photoacoustic image information may be generated by 1st 2D scanning from the first scan line to the n-th scan line, and, without moving the photoacoustic probes 11 and 21 to the first scan line, ultrasonic image information may be generated by second 2D scanning from the n-th scan line to the first scan line at the position where the first 2D scanning is finished. In this case, after reversing the order of the ultrasonic image information, the ultrasonic image information is combined with the photoacoustic image information to generate a combined image.

As another embodiment, after generating photoacoustic image information and ultrasonic image information for one scan line, 2D scanning may also be performed while generating photoacoustic image information and ultrasonic image information for each scan line while moving the photoacoustic probes 11 and 21 in the second direction.

Here, for the n-th scan line, photoacoustic image information may be received while moving the photoacoustic probes 11 and 21 in the first direction and ultrasonic image information may be received while moving the photoacoustic probes 11 and 21 in a direction opposite to the first direction, and for the (n+1)-th scan line, photoacoustic image information may be received while moving the photoacoustic probes 11 and 21 in the first direction and ultrasonic image information may be received while moving the photoacoustic probes 11 and 21 in the direction opposite to the first direction.

As another embodiment, photoacoustic image information may be generated by 1st 2D scanning from the first scan line to the last scan line, and, without moving the photoacoustic probes 11 and 21 to the first scan line, ultrasonic image information may be generated by second 2D scanning from the last scan line to the first scan line at the position where the first 2D scanning is finished. In this case, after reversing the order of the ultrasonic image information, the ultrasonic image information is combined with the photoacoustic image information to generate a combined image.

Meanwhile, for the same n-th scan line, scanning for generating photoacoustic image information may be performed once, and scanning for generating ultrasonic image information may be performed once. In this case, the second-direction motion of the photoacoustic probes 11 and 21 can be minimized, thereby maximizing the overall 2D scanning time and efficiency.

That is, while moving in the first direction from the first end to the second end of the same n-th scan line, photoacoustic image information may be generated by a positive first-direction motion of the photoacoustic probes 11 and 21 and, while moving in the first direction from the first end to the second end of the same n-th scan line, without moving the photoacoustic probes 11 and 21 in the second direction, ultrasonic image information may be generated by the positive first-direction motion of the photoacoustic probes 11 and 21.

In this case, photoacoustic image information and ultrasonic image information can be acquired at the same location of the to-be-examined object.

As another embodiment, while moving from the first end to the second end of the n-th scan line in the first direction, photoacoustic image information may be generated by the positive (+) first-direction motion of the photoacoustic probes 11 and 21, and, while moving from the second end of the n-th scan line in the direction opposite to the first direction of the first end, without moving the photoacoustic probes 11 and 21 in the second direction, ultrasonic image information may be generated by a reverse first-direction motion (negative (−) first-direction motion) of the photoacoustic probes 11 and 21.

In this case, in order to generate ultrasonic image information, without the need to move the photoacoustic probes 11 and 21 to the first end again after the first scanning in the first direction, for the same n-th scanning line, a second scanning is performed to generate ultrasonic image information at the second end. Therefore, the first- and second-direction motions of the photoacoustic probes 11 and 21 can be minimized, thereby increasing the overall 2D scanning time and efficiency.

As another embodiment, while the photoacoustic probes 11 and 21 move in the first direction, which is a direction from the first end to the second end of the n-th scan line, laser pulse output and ultrasonic output may be alternately performed in the photoacoustic probes 11 and 21. Accordingly, a photoacoustic image and an ultrasonic image can be acquired by performing the first-direction scanning once.

Here, compared to an embodiment in which photoacoustic scan and ultrasonic scan are separately performed, linear encoder pulse signals are set to be a ½ interval, and photoacoustic output and ultrasonic output can be controlled to be alternately performed while performing scanning once for the n-th scan line. In this case, both a photoacoustic image and an ultrasonic image can be acquired by scanning once in each of the first and second directions.

To this end, 3D image information for the to-be-examined object is generated by 2D scanning of the photoacoustic probes 11 and 21 once, and the first ultrasonic input and the second ultrasonic input may be alternately performed within each scanning line.

Here, while moving the photoacoustic probes 11 and 21 in the first direction for the n-th scan line, photoacoustic image information and ultrasonic image information may be alternately received, and, while moving the photoacoustic probes 11 and 21 in a direction opposite to the first direction with respect to the n+1th scan line, photoacoustic image information and ultrasonic image information may be alternately received. In this case, photoacoustic image and ultrasonic image can be acquired simultaneously while moving the photoacoustic probes 11 and 21 as little as possible.

The combined photoacoustic/ultrasonic image input apparatus 1 may include an output selector that selects laser pulse output or ultrasonic output. The output selector may select to output a laser pulse output through the laser output unit 11 or output an ultrasonic pulse through the ultrasonic probe 21 according to the output selection signal generated by the main controller 40.

The output selector may be included inside the pulse signal generator 60, and may be controlled so that the reference pulse signal is output to the laser generator 10 or the trigger controller 50 according to the output selection signal input from the main controller 40.

Here, the main controller 40 may generate and output an output selection signal of laser output or ultrasonic output, and the pulse signal generator 60 may output the reference pulse signal to the trigger controller 50 for ultrasonic output or the laser generator 10 for laser output according to the output selection signal.

Meanwhile, the combined photoacoustic/ultrasonic image input apparatus 1 may further include a second linear encoder 70 that generates a linear encoder pulse signal corresponding to the second-direction linear motion information of the photoacoustic probes 11 and 21.

In addition, the combined photoacoustic/ultrasonic image input apparatus 1 may further include a memory for storing plane coordinate values of the photoacoustic probe 11, 12, determined by the first-direction linear motion information and the second-direction linear motion information, photoacoustic image information at the plane coordinate values, and ultrasonic image information for the plane coordinate values of the photoacoustic probe 11, 12 and the to-be-examined object at the plane coordinate values.

The combined photoacoustic/ultrasonic image input apparatus 1 may be implemented by an optical-resolution type apparatus 2 for inputting a combined photoacoustic/ultrasonic image, which is schematically shown in FIG. 2.

In the optical-resolution type combined photoacoustic/ultrasonic image input apparatus 2, the laser pulse generated by the laser generator may pass through a half wave plate (HWP), a variable beam splitter/attenuator (VBA), and a fiber coupler (FC), and may be transmitted through a polarization-maintaining single-mode fiber (PM-SMF) and irradiated to the to-be-examined object by the laser output unit. Here, the laser output unit may include a band pass filter (BPF), first and second objective lenses (OL1 & OL2), and a corrective lens (CL).

Meanwhile, the ultrasonic pulse generated by the ultrasonic transceiver (pulser/receiver (PR)) may be irradiated to the to-be-examined object through an ultrasonic probe (SFT), and the first ultrasonic input and the second ultrasonic input generated from the to-be-examined object may be input to the ultrasonic transceiver (pulser/receiver (PR)) through the ultrasonic probe (SFT). The ultrasonic image signal input through the ultrasonic transceiver may be input to a signal processing device corresponding to the A/D converter 30 and the main controller 40 to generate a photoacoustic image signal and an ultrasonic image signal, and a combined image may be generated.

The laser pulse output and the ultrasonic pulse output may be irradiated to the to-be-examined object through an optical-acoustic beam combiner (OABC), or the first ultrasonic input and the second ultrasonic input generated from the to-be-examined object may be input to the ultrasonic probe (SFT) through an optical-acoustic beam combiner (OABC).

The ultrasonic pulse output, a first ultrasonic input, and a second ultrasonic input may output or input by using as a medium water contained in a water dish, and a plastic membrane (PMB) may be disposed on the upper and lower surfaces of the water dish.

Meanwhile, the photoacoustic probe may be transferred in the first direction and/or the second direction by a transfer stage when scanning the to-be-examined object, and the operation of the transfer stage may be controlled by a motion controller.

Alternatively, in a method for inputting a combined photoacoustic/ultrasonic image according to an embodiment of the present disclosure, a combined photoacoustic-ultrasonic image may be acquired by using the combined photoacoustic/ultrasonic image input apparatus 1 by the above-described method. Here, according to the method for inputting a combined photoacoustic/ultrasonic image, the combined photoacoustic-ultrasonic image can be acquired by the method for generating a combined image by a photoacoustic image signal, indicated by a timing diagram shown in FIG. 3 and the method for generating a combined image by an ultrasonic image signal, indicated by a timing diagram shown in FIG. 4.

As described above, according to the present disclosure, by generating a photoacoustic image and ultrasonic image for the inside of a to-be-examined object, respectively, while moving a single photoacoustic probe at high speed, a combined image in which the photoacoustic image and the ultrasonic image are combined may be generated by using only the photoacoustic probe even without a separate ultrasonic module.

In addition, since structure-related information and blood vessel-related information for the inside of a human body are simultaneously displayed in one image, doctors can helped to make a more accurate diagnosis in diagnosing diseases of the inside of the human body.

The technical idea of the present disclosure has been hitherto described through the disclosure of a preferred embodiment of the present disclosure that guarantees the specificity of the idea. A person having ordinary knowledge in the technical field to which the present disclosure belongs will be able to understand that preferred embodiments may be implemented in a modified form within a range that does not deviate from the technical spirit (essential features) of the present disclosure. Therefore, the embodiments disclosed herein should be considered from a descriptive point of view rather than a limiting point of view, and the scope of rights of the present disclosure should be construed as including not only the matters disclosed in the claims but also all differences being within the range equivalent thereto.

What is claimed is:

1. An apparatus for inputting a combined photoacoustic/ultrasonic image, which generates three-dimensional (3D) image information for a to-be-examined object by performing two-dimensional (2D) scanning on the to-be-examined object by a first-direction linear motion of a photoacoustic probe and a second-direction linear motion that is substantially perpendicular to the first-direction linear motion, the apparatus comprising:
a photoacoustic probe that outputs a laser pulse output or an ultrasonic output to a to-be-examined object, receives a first ultrasonic input from the to-be-examined object by the laser pulse output, and receives a second ultrasonic input from the to-be-examined object by the ultrasonic output;
an ultrasonic transceiving unit that generates an ultrasonic output signal for generating the ultrasonic output, receives the first ultrasonic input and the second ultrasonic input, and generates a photoacoustic image signal and an ultrasonic image signal, respectively;
an analog/digital (A/D) converter that receives the photoacoustic image signal and the ultrasonic image signal to convert the same into digital image signals; and
a main controller that receives the digital image signals, generates photoacoustic image information and ultrasonic image information for the to-be-examined object, and combines the photoacoustic image information and ultrasonic image information to generate a combined photoacoustic/ultrasonic image.

2. The apparatus as claimed in claim 1, wherein the photoacoustic probes include a laser output unit that outputs the laser pulse output to the to-be-examined object, and an ultrasonic probe that outputs the ultrasonic output to the to-be-examined object and receives the first ultrasonic input and the second ultrasonic input, respectively.

3. The apparatus as claimed in claim 1, comprising:
a pulse signal generator that generates and outputs reference pulse signals at a set interval;
a first linear encoder that generates first-direction linear motion information of the photoacoustic probe;
a laser generator that outputs a laser pulse at a set interval to the to-be-examined object according to a reference pulse signal and the first-direction linear motion information; and
a trigger controller that generates an output trigger signal at a set interval according to the reference pulse signal and the first-direction linear motion information.

4. The apparatus as claimed in claim 3, wherein the ultrasonic transceiving unit generates the photoacoustic image signal and ultrasonic image signal corresponding to the first-direction linear motion information according to the output trigger signal, respectively.

5. The apparatus as claimed in claim 2, wherein the ultrasonic probe outputs an ultrasonic output corresponding to an output trigger signal generated by the trigger controller and receives the ultrasonic input corresponding to the first-direction linear motion information according to the output trigger signal.

6. The apparatus as claimed in claim 1, wherein in the main controller, photoacoustic image information for the to-be-examined object is generated by sequentially combining the photoacoustic digital image signal with the photoacoustic image information corresponding to each trigger pulse of the output trigger signal in a positive direction or a negative direction of the first direction in units of photoacoustic digital image scan lines, and ultrasonic image information for the to-be-examined object is generated by sequentially combining the ultrasonic digital image signal with the ultrasonic image information corresponding to each trigger pulse of the output trigger signal in the positive or negative direction of the first direction in units of scan lines.

7. The apparatus as claimed in claim 1, wherein the photoacoustic image information is generated by first 2D scanning from a first scan line to an n-th scan line, and the ultrasonic image information is generated by second 2D scanning from the first scan line to the n-th scan line.

8. The apparatus as claimed in claim 1, wherein the photoacoustic image information is generated by first 2D scanning from a first scan line to an n-th scan line, and the ultrasonic image information is generated by second 2D scanning from the n-th scan line to the first scan line.

9. The apparatus as claimed in claim 1, wherein:
while moving in a first direction from a first end to a second end of the n-th scan line, the photoacoustic image information is generated by a positive (+) first-direction motion of the photoacoustic probe; and
without moving the photoacoustic probe in a second direction, while moving in the first direction from the first end to the second end of the n-th scan line, the ultrasonic image information is generated by the positive (+) first-direction motion of the photoacoustic probe.

10. The apparatus as claimed in claim 1, wherein: while moving in the first direction from the first end to the second end of the n-th scan line, the photoacoustic image information is generated by the positive (+) first-direction motion of the photoacoustic probe; and
without moving the photoacoustic probe in the second direction, while moving in a direction opposite to the first direction from the second end to the first end of the n-th scan line, the ultrasonic image information is generated by a reverse motion (negative (−) first-direction motion) to the first-direction motion of the photoacoustic probe.

11. The apparatus as claimed in claim 1, further comprising an output selector for selecting the laser pulse output or the ultrasonic output so as to be output.

12. The apparatus as claimed in claim 3, further comprising an output selector for selecting a reference pulse signal to be output to the trigger controller or the laser generator.

13. The apparatus as claimed in claim 3, wherein the main controller generates and outputs an output selection signal, and the pulse signal generator outputs the reference pulse signal to the trigger controller or the laser generator according to the output selection signal.

14. The apparatus as claimed in claim 1, wherein while moving the photoacoustic probe in the first direction, which is a direction from the first end to the second end of the n-th scan line, the laser pulse output and the ultrasonic output are alternately performed in the photoacoustic probe.

15. The apparatus as claimed in claim 1, wherein 3D image information for the to-be-examined object are generated by one-time 2D scanning of the photoacoustic probe, and the first ultrasonic input and the second ultrasonic input are alternately performed within each scanning line.

16. The apparatus as claimed in claim 1, further comprising:
a second linear encoder for generating the second-direction linear motion information of the photoacoustic probe; and
a memory for storing plane coordinate values of the photoacoustic probe, determined by the first-direction linear motion information and the second-direction linear motion information, photoacoustic image information at the plane coordinate values, and ultrasonic image information for the plane coordinate values of the photoacoustic probe and the to-be-examined object at the plane coordinate values.

17. The apparatus as claimed in claim 1, wherein, with respect to the to-be-examined object, the photoacoustic image information is generated by 2D scanning while allowing the photoacoustic probe to alternately perform the first-direction motion and the second-direction motion from start coordinates to end coordinates, and, after completing the 2D scanning, the ultrasonic image information is generated by the 2D scanning while allowing the photoacoustic probe to alternately perform the first-direction motion and the second-direction motion from the start coordinates to the end coordinates.

18. The apparatus as claimed in claim 1, wherein: with respect to the n-th scan line, the photoacoustic image information is received while moving the photoacoustic probe in the first direction, and the ultrasonic image information is received while moving the photoacoustic probe in a direction opposite to the first direction, and, with respect to an (n+1)-th scan line, the photoacoustic image information is received while moving the photoacoustic probe in the first direction, and the ultrasonic image information is received while moving the photoacoustic probe in a direction opposite to the first direction.

19. The apparatus as claimed in claim 1, wherein: with respect to the n-th scan line, the photoacoustic image information and the ultrasonic image information are alternately received while moving the photoacoustic probe in the first direction; and, with respect to an (n+1)-th scan line, the photoacoustic image information and the ultrasonic image information are alternately received while moving the photoacoustic probe in a direction opposite to the first direction the photoacoustic image information.
